(19) United States
(12) Patent Application Publication
(10) Pub. No.: US 2009/0130208 A1
(43) Pub. Date: May 21, 2009

(54) Modified release niacin formulations

(75) Inventors: Subraman Rao Cherukuri, Vienna, VA (US); Revanth Babu Mutyala, Frederick, MD (US); Venkat Ravella, Gandhinagar (IN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Capricorn Pharma Inc., Frederick, MD (US)

(21) Appl. No.: 12/275,634

(22) Filed: Nov. 21, 2008

Related U.S. Application Data
(60) Provisional application No. 61/003,997, filed on Nov. 21, 2007.

(51) Int. Cl. A61K 9/24; A61P 9/10

(52) U.S. Cl. 424/472

(57) ABSTRACT

Modified release niacin formulations, methods for making the formulations, and methods of using the formulations.

### CROSS-REFERENCES TO RELATED APPLICATIONS

This application claims the benefit of U.S. Provisional Application Ser. No. 61/003,997, filed Nov. 21, 2007, the contents of which are expressly incorporated herein.

### FIELD OF THE INVENTION

The invention is directed to modified release niacin formulations, methods of making the formulations, and methods of using the formulations.

### BACKGROUND OF THE INVENTION

Niacin has long been known to provide beneficial cholesterol lowering effects for treatment of disorders such as hyperlipidemia. Immediate release niacin formulations typically require multiple daily doses to be effective. Modified release niacin formulations are desirable because they can achieve better control of hyperlipidemia for a longer period of time compared to immediate release formulations. Immediate release formulations often require multiple dosing in a single day. Therefore, modified release niacin formulations are more convenient and result in better patient compliance.

Modified release niacin formulations are sold commercially under the brand name Niaspan®. The Federal Food & Drug Administrations (“FDA's”) Approved Drug Products with Therapeutic Equivalents (“the Orange Book”) lists U.S. Pat. Nos. 6,080,428; 6,129,930; 6,406,715; 6,469,035; 6,676,967; 6,746,691; 6,818,229; 7,011,848 in connection with Niaspan®.

### SUMMARY OF THE INVENTION

The invention is directed to modified release niacin formulations. The modified release niacin compositions comprises:

a plurality of granules comprising a therapeutically effective amount of niacin, a pharmaceutically acceptable binder, and an excipient,

wherein the granules are compressed together to form a core and the core is coated with a release-modifying coating.

The invention is further directed to a method of making the modified release niacin formulations and methods of administering niacin to a subject in need thereof by administering the modified release niacin formulation to the subject.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 provides a graphical representation of the in-vitro dissolution profile of the 500 mg coated tablet of Example 5.

FIG. 2 provides a graphical representation of the in-vitro dissolution profile of the Niacin 1000 mg/lovastatin 20 mg coated tablet of Example 8.

### DETAILED DESCRIPTION OF THE INVENTION

The invention is directed to modified release niacin formulations, methods of making the formulations, and methods of using the formulations.

### Definitions

In describing and claiming the present invention, the following terminology will be used in accordance with the definitions set forth below.

As used herein, the singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” and “pro-drug” are expressly encompassed by the present definition as many drugs and prodrugs are known to have specific physiologic activities. These terms of art are well-known in the pharmaceutical and medicinal arts.

As used herein, “subject” refers to a mammal that may benefit from the administration of a composition or a method of this invention. Examples of subjects include, but are not limited to, humans, horses, pigs, cattle, dogs, cats, rabbits, and aquatic mammals.

As used herein, “niacin” refers to a compound commonly known as 3-pyridinecarboxylic acid and related compounds, such as salts, acid addition salts, prodrugs, isomers and metabolites thereof, as well as mixtures thereof as dictated by the context of its use. As such, inherent support for all known individual specific compound names in view of the above-recited definition, is considered to be included herein, though they may not each be expressly recited.

As used herein, “blood level” may be used interchangeably with terms such as “blood plasma concentration,” “plasma level,” “plasma concentration,” “serum level,” “serum concentration,” “serum blood level,” and “serum blood concentration.”

As used herein, “oral dosage form” and the like, refers to a formulation that is ready for administration to a subject through the oral route of administration. Examples of known oral dosage forms, include without limitation, tablets, capsules, caplets, powders, pellets, granules, etc. Such formulations also include multilayered tablets wherein each layer contains a different drug. In some aspects, powders, pellets, and granules may be coated with a suitable polymer or a conventional coating material to achieve, for example, greater stability in the gastrointestinal tract, or to achieve a desired rate of release. Moreover, capsules containing a powder, pellets or granules may be further coated. Tablets and caplets may be scored to facilitate division for dosing. Alternatively, the dosage forms of the present invention may be unit dosage forms wherein the dosage form is intended to deliver one therapeutic dose per administration.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors, such as, for example, absorption into the body through the oral/gastrointestinal tract, which can vary depending on the individual, may affect the ability of a substance to perform its intended task. Therefore, an “effective amount” or a “therapeutically effective amount” may be dependent, in some instances, on such biological factors. Further, while the achievement of a therapeutic effect may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert, pharmaceutically acceptable material that has substantially no biological activity, and makes up a substantial part of the formulation.

The term “admixed” means combined and mixed with. For example, a drug and/or other ingredients can be admixed with a carrier to provide a drug a composition wherein the drug is dissolved, dispersed, or suspended in the carrier. In some cases, the drug may be uniformly admixed with the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. In various aspects “substantially” can mean at least 90%, at least 95%, at least 98%, at least 99%, at least 99.5%. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof. In various aspects the phrase “substantially free of” can mean less than 10%, less than 5%, less than 2%, less than 1%, or less than 0.5%. For example, the phrase “a composition substantially free of particles” can mean a composition that has less than 10%, less than 5%, less than 2%, less than 1%, or less than 0.5% of particles.

The term “modified release” or “release-modifying,” as used herein, refers to a drug release profile that is different from the drug release profile of an immediate release dosage form. This release profile may be measured in terms of the dissolution of the drug in a dissolution medium at a specified time(s). In one aspect, the release profile is measured under the conditions specified in the USP, e.g., where the pH is maintained at 1.2 for 2 hours, followed by a pH of 6.8 for the rest of the time. In another aspect, the release profile is measured at a pH of 1.2 for the entire period of measurement. Typically, with an immediate release dosage form, more than about 80% of the drug is released from the dosage form in vitro within about 2 hrs under specified conditions. A modified release dosage form would release the drug more slowly than the immediate release dosage form under the same conditions. Examples of such modified release include sustained release, slow-release, delayed-release, pulsatile release etc., which terms are generally known in the art and to the extent they mean a release other than an immediate release.

As used herein, the term “about” is used to provide flexibility to a numerical range endpoint by providing that a given value may be “a little above” or “a little below” the endpoint. In one embodiment, the term “about” means±10%, ±5%, ±2%, ±1%, or ±0.5%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent to any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The term “core,” as used herein, means a plurality of particles that have been compressed together to form a larger structure. For example, granules can be compressed together to form a tablet or a mini-tablet. A tablet is a structure having a size of greater than 7 mm. A mini-tablet is a structure having a size ranging from 3 to 7 mm.

The term “granules,” as used herein, has its art recognized meaning, i.e., a combination of more than one substances (for example, a pharmaceutically active compound one or more excipients) in a particle. The granule can be, for example, a bead or a spheroid. Granules can be made using art recognized methods including, but not limited to, wet granulation, dry granulation, extrusion, spheronization, spray-drying, and congealing.

The phrase “release-modifying coating,” as used herein, means a coating that, when applied to a particle or core containing a pharmaceutically active compound, provides a coated particle or core that releases the pharmaceutically active compound more slowly than an identical particle or core but without the coating when the release rates of the coated and uncoated particle are compared under the same conditions.

### The Modified Release Niacin Formulations

The modified release niacin formulations comprise:

a plurality of granules comprising a therapeutically effective amount of niacin, a pharmaceutically acceptable binder, and an excipient,

wherein the granules are compressed together to form a core and the core is coated with a release-modifying coating.

The core can be, for example, a tablet or a minitablet.

The amount of niacin per dosage form can be, as stated conventionally, from about 25.0 mg up to about 2000 mg, including specific intermediate amounts such as 375 mg, 500 mg, and 750 mg.

Any binder known to those skilled in the art can be used. Illustrative binders include, but are not limited to, vinyl polymers, such as polyvinylpyrrolidone, polyvinyl alcohol, and the like; cellulosic polymers, such as hydroxypropylmethyl cellulose (HPMC), hydroxyethyl cellulose (HEC), hydroxypropyl cellulose (HPC), and the like; acrylic polymers and copolymers such as methacrylic acid polymers, ethyl acrylate-methylmethacrylate copolymers, and the like; natural or synthetic gums, such as guar gum, arabic gum, xanthan gum, and the like; proteins or carbohydrates, such as gelatin, pectin, and the like; and mixtures thereof. In one embodiment, ethylcellulose and/or polyvinylpyrrolidone is the binder. In one embodiment, polyvinylpyrrolidone is the binder. In one embodiment, the binder is selected from ethylcellulose, hydroxypropylmethyl cellulose, or a mixture thereof.

One of ordinary skill in the art will readily be able to determine the amount of binder to use. Typically, the binder(s) is present in an amount of about 0.01% to about 70.00% by weight of the granules. In one embodiment, the binder(s) is present in an amount of about 0.01% to about 30.00% by weight of the granules

Any pharmaceutically acceptable excipient known to those skilled in the art can be used. In one embodiment, the pharmaceutically acceptable excipient is selected from the group consisting of: sugars, carbohydrates, polyols, celluloses, microcrystalline cellulose, mono-, di-, and tri-basic calcium phosphates, starch, sodium starch glycolate, crospovidone, croscarmellose sodium, magnesium stearate, lactose, stearic acid, maleic acid, a wax, colloidal silicon dioxide, talc, gelatin, polyethylene glycol, titanium dioxide, glyceryl behenate, fats, emulsifiers, and mixtures thereof.

One of ordinary skill in the art will readily be able to determine the amount of excipients to use. Typically, the excipient(s) are present in an amount sufficient to reach 100% with any individual excipient typically in an amount ranging from about 0.01 to about 95.0% by weight of the granules.

The granules can be prepared by any method known to those skilled in the art. Suitable methods include wet granulation and dry granulation. Extrusion and spheronization may also be used to provide the granules. In one embodiment, the granules are obtained by wet granulation using a non-aqueous granulation solvent. Suitable non-aqueous solvents include, but are not limited to ethanol, isopropanol, acetone, methanol, and mixtures thereof. In another embodiment, the granule are obtained by wet granulation using water as the granulating solvent.

The granules can be compressed together to provide a core using methods well known to those skilled in the art. The core can be, for example, a tablet, a mini-tablet, or a bead.

The core can be coated with the release-modifying coating using methods well known to those skilled in the art. For example, the release-modifying coating can be a release-modifying polymer that provides modified release through diffusion or pH-dependency. Illustrative release-modifying polymers include, but are not limited to, cellulose-based polymers, acrylate polymers, and waxes. Illustrative cellulose-based polymers include, but are not limited to, ethylcellulose, propylcellulose, hydroxymethylpropyl cellulose, hydroxypropyl cellulose. Illustrative acrylate polymers include, but are not limited to, polymers of methylmethacrylate and polymers of methacrylate.

In one specific aspect, the release-modifying coating comprises ethylcellulose. In another aspect, the release-modifying coating consists essentially of ethylcellulose. In another aspect, the release-modifying coating consists of ethylcellulose.

The coating composition may also include a plasticizer such as triethylene acetate, triacetene, glycerol, diethyl phthalate, and the like.

The amount of the release-modifying coating typically ranges from about 0.5% to about 30% by weight of the uncoated tablet or granule. In some aspects, the coating may range from about 0.5% to about 25%; from about 0.5% to about 20%; from about 0.5% to about 15%; from about 0.5% to about 10%; from about 0.5% to about 5%; from about 0.5% to about 2%, from about 0.5% to about 1%, or from about 0.5% to about 0.75% by weight of the uncoated tablet or granule.

In one aspect, the modified release niacin formulation provides the following dissolution profile:

about 5% to about 25% of the drug is released by 2 hrs;

about 20% to about 40% of the drug is released by 4 hrs;

about 30% to about 50% of the drug is released by 6 hrs;

about 40% to about 70% of the drug is released by about 12 hours;

about 50% to about 80% of the drug is released by about 16 hours; and

about 60% to about 90% of the drug is released by about 20 hours,

when dissolution is determined using 900 ml of an aqueous medium, in a dissolution apparatus using a basket, operated at about 37° C., and being stirred at a speed of 100 rpm.

In another aspect, the modified release niacin formulation provides the following dissolution profile:

about 5% to about 25% of the drug is released by 2 hrs;

about 20% to about 40% of the drug is released by 4 hrs;

about 30% to about 50% of the drug is released by 6 hrs;

about 50% to about 80% of the drug is released by about 12 hours;

about 60% to about 90% of the drug is released by about 16 hours; and

about 70% to about 100% of the drug is released by about 20 hours,

when dissolution is determined using 900 ml of aqueous medium, in a dissolution apparatus using a basket, operated at about 37° C., and being stirred at a speed of 100 rpm.

In an alternate aspect, the modified release niacin formulation provides the following dissolution profile:

about 5% to about 25% of the drug is released by 2 hrs;

about 20% to about 50% of the drug is released by 4 hrs;

about 30% to about 60% of the drug is released by 6 hrs;

about 50% to about 80% of the drug is released by about 12 hours;

about 60% to about 85% of the drug is released by about 16 hours; and

about 70% to about 100% of the drug is released by about 20 hours,

when dissolution is determined using 900 ml of aqueous medium, in a dissolution apparatus using a basket, operated at about 37° C., and being stirred at a speed of 100 rpm.

The invention is further directed to a method of making the modified release niacin formulations. The modified release niacin formulations are made by

a) combining niacin, a binder, and a pharmaceutically acceptable excipient to provide a niacin-containing mixture,

b) granulating the niacin-containing mixture to provide niacin-containing granulates;

c) compressing said niacin-containing granulates to provide a niacin-containing core; and

d) coating the niacin-containing core with a release-modifying coating to provide a release-modified coated core.

In one embodiment, the process further involves

e) coating the release-modified coated core with a second coating, wherein the second coating comprises a pharmaceutically active agent.

In one embodiment, the binder is present in an amount ranging from about 1% to about 10% by weight of the granules.

In one embodiment, the release-modifying coating material is present in an amount ranging from about 0.5% to about 10% by weight of the uncoated tablet.

In one specific embodiment of the invention, the method for making the modified release niacin formulations involves:

a) combining niacin, a binder, and a pharmaceutically acceptable excipient to provide a niacin-containing mixture,

b) granulating the niacin-containing mixture to provide niacin-containing granulates;

c) compressing said niacin-containing granulates to provide a niacin-containing core; and

d) coating the niacin-containing core with a release-modifying coating,

to provide the modified release niacin formulation,
wherein the niacin is present in an amount ranging from about 250 mg to about 1000 mg and the release-modifying coating is present in an amount ranging from 0.5% to about 10% by weight of the modified release niacin formulation.

Combining the niacin, binder(s), and excipient(s) to provide the niacin-containing mixture can be accomplished by using high shear granulators (mixers, blenders, etc) or rapid mixer granulators. The homogenous mixture may be then processed into granules, preferably by wet granulation processes (i.e., using water as the granulating medium) as is known in the art. Granulation may be performed using art-known equipment such as high shear granulators, fluid bed granulators, etc. These granules are then optionally dried. The drying process may provide certain advantages such as improvements in content uniformity, ease of handling, etc.

Alternatively, the niacin, binder(s), and excipient(s) may be granulated with a non-aqueous solvent. Examples of non-aqueous solvents include, but are not limited to, ethanol, isopropanol, acetone, methanol, and mixtures thereof.

Suitable granulation solvents are those that are capable of substantially completely solubilizing the specific binder(s) and are pharmaceutically and biologically acceptable for ingestion. One of ordinary skill in the art will readily be able to determine what is a suitable granulation solvent for a binder. Water is currently the preferred granulation solvent. Other examples of suitable solvents, however, will be appreciated by those skilled in the art and are contemplated by the methods of the present invention.

The solution of the binder should be of sufficient viscosity to enable the wetting of the dry powder mix (i.e., the niacin and excipients) by any suitable wetting technique known to those skilled in the art. For example, the dry powder mix may be wetted with the binder solution by rotating the dry powder mix in a bath containing the binder solution. The dry powder mix may be suitably wetted by manual application of the binder solution by layering the binder solution over the powder as the powder is rotating in a conventional coating pan. Alternatively, the dry powder mix may be wetted by spraying the binder solution on the dry powder mix. In one aspect, the wetting step is carried out using conventional automated pan coating equipment wherein the dry powder mix is sprayed with the binder solution while rotating in the pan. However, the granulation step can be accomplished in one single pot (i.e., the rapid mixer granulator) without the need to transfer the dry mix into a separate granulator, if it is conducted in the rapid mixer granulator itself. Rather than using a solution of the binder in the granulating solvent, it is also possible to use a dispersion of the binder in the granulating solvent.

Suitable excipients are known to those skilled in the art and include, but are not limited to, lubricants, flow promoting agents, plasticizers, anti-sticking agents, natural and synthetic flavorings, and natural and synthetic colorants.

The amount of “excipient” typically ranges from about 0.01% to about 95.00% by weight of the granules. In some other aspects, the excipient amount may have the following ranges: from about 0.01% to about 12%; from about 0.01% to about 10%; from about 0.01% to about 8%; from about 0.01% to about 7%; from about 0.01% to about 6%; from about 0.01% to about 5%; from about 2% to about 15%; from about 2% to about 12%; from about 2% to about 10%; from about 2% to about 8%; from about 2% to about 6%; from about 2% to about 5%; from about 2% to about 4%; from about 3% to about 15%; from about 3% to about 12%; from about 3% to about 10%; from about 3% to about 8%; from about 3% to about 6%; from about 3% to about 5%; from about 4% to about 12%; from about 4% to about 10%; from about 4% to about 8%; from about 4% to about 6%; from about 5% to about 15%; from about 5% to about 12%; from about 5% to about 10%; from about 5% to about 8%; or from about 5% to about 7%.

In some aspects, the particle size of the niacin-containing granules has the following distribution:

about less than 40% of the granules are retained by a #40 mesh;

about less than 70% of the particles are retained by a #60 mesh;

about less than 90% of the particles are retained by a #80 mesh; and

about 100% of the particles are retained by a #200 size mesh.

Alternatively, in some aspects, the particle size of the niacin-containing granules has the following distribution:

about less than 60% of the granules are retained by a #40 mesh;

about less than 80% of the particles are retained by a #60 mesh;

about less than 90% of the particles are retained by a #80 mesh; and

about 100% of the particles are retained by a #200 size mesh.

Alternatively, in some aspects, the particle size of the niacin-containing granules has the following distribution:

about less than 60% of the granules are retained by a #40 mesh;

about less than 90% of the particles are retained by a #60 mesh; and

about 100% of the particles are retained by a #200 size mesh.

An illustrative granulation process involves the following steps. Niacin is sifted through a #20 sieve. Excipients may be sifted through a #40 sieve. The sifted materials are loaded into a rapid mixer granulator (RMG) and rapidly mixed for about 5-20 minutes. To this dry mix is added a solution or dispersion of the binder in an aqueous or non-aqueous solvent. The binder may be any binder that is known the art. In one specific embodiment, the binder is polyvinylpyrrolidone K-30 and the non-aqueous solvent is isopropyl alcohol.

This niacin-containing granulates are then optionally dried to substantially remove any residual solvents. The niacin-containing granulates are then compressed into a core.

In one embodiment, the niacin-containing granulates are coated with a release-modifying coating and then compressed into a core.

The resulting core (comprising either coated or uncoated granulates) is then coated with the release-modifying coating. The release-modifying coating substantially surrounds the core. The release-modifying coating that coats the granules and the release-modifying coating that coats the core can be the same or different and may be present in the same or different amounts. Coating may be accomplished by either aqueous or non-aqueous solvent-based techniques. The coating equipment is known in the art. For example, conventional coating pans or automatic fluid bed coaters may be used.

The invention provides a niacin containing dosage form prepared according to the methods described herein.

The invention is further directed to an article of manufacture comprising the modified release niacin formulation accompanying labeling and packaging to enable the article of manufacture to be shipped interstate.

The invention also provides modified release formulations of niacin in combination with other pharmaceutically active agents. Examples of such additional agents include: cardiovascular agents and non-steroidal anti-inflammatory agents. Examples of cardiovascular agents include: ACE inhibitors, antihyperlipidemic agents, calcium channel blockers, beta-blockers and antiplatelet agents. Anti-hyperlipidemic agents include: statins, bile acid sequestrants, fibrates and related compounds. Examples of statins include: fluvastatin, simvastatin, atorvastatin, etc. Examples of bile acid sequestrants include: coesevelam, cholestyramine, and colestipol. Examples of fibrates and related compounds include: fenofibrate, gemfibrozil, etc. Examples of anti-platelet agents include: dipyridamole, aspirin, clopidogrel, among others. Examples of non-steroidal agents include: aspirin, diclofenac, ibuprofen, ketoprofen, piroxicam, etc. It should be noted that the additional ingredient may be present in an immediate release form, in a modified release form, or in a pulsatile release form, or in a fast disintegrating form. The additional agent may be present in an intimate mixture with the niacin or separated by an additional barrier layer.

In one embodiment, the coated niacin-containing core is further coated with another pharmaceutically active agent and then with a protective coating. In one embodiment, the coated niacin-containing core is coated with HMG-CoA reductase inhibitor and then with a protective coating. In another embodiment, the coated niacin-containing core is coated with lovastatin and then with a protective coating. Suitable dosage units include tablets, minitablets capsules, caplets and beads. Typically, the protective coating allows for immediate release of the other pharmaceutically active agent. The other pharmaceutically active agent can be coated on the coated niacin-containing core using methods known to those skilled in the art. Similarly, the protective coating can be applied using methods known to those skilled in the art.

In another embodiment, the granules coated with a release-modifying coating or the coated niacin containing core are used to fill a conventional hard or soft-gelatin capsule. In another embodiment, the capsules are coated with a release-modifying coating. Encapsulation within a soft-gelatin capsule is also achievable using conventional techniques.

In another embodiment, the granules coated with a release-modifying coating or the coated niacin containing core (for example, a minitablet) are further coated with another pharmaceutically active agent and then with a protective coating. The resulting granules (or core) are then used to fill a capsule to provide the dosage form.

In another embodiment, the invention involves coating niacin containing granules (which, for example, can be beads or spheroids) or niacin containing minitablets with a release-modifying coating and also preparing granules containing another pharmaceutically active agent (which can be prepared by methods similar to those used to prepare the niacin-containing granules) and then coating the granules containing the other pharmaceutically active agent with a release-modifying coating or simply a protective coating (i.e., that does not substantially modify release). The coated niacin containing granules and the coated granules containing the other pharmaceutically active agent are then combined in a capsule to provide a combination dosage form.

The invention further relates to a method of administering niacin to a subject comprising administering the modified release niacin formulation to the subject. In one embodiment the subject is a human.

### EXAMPLES

Without further elaboration, it is believed that one skilled in the art using the preceding description can utilize the invention to the fullest extent. The following examples are illustrative only, and not limiting of the disclosure in any way whatsoever.

All percentages are in percent by weight of the formulation unless otherwise indicated. Dissolution tests are carried out according to the standard procedures set forth in the United States Pharmacopoeia for testing dissolution from tablets.

### Example 1

A modified release niacin formulation was prepared from a niacin-containing mixture having the following composition:

| Sl. No. | Ingredients | Qty. for trial | |
|---|---|---|---|
| 1 | Niacin | 275.00 | g |
| 2 | MCC (Powder) I.G. | 68.75 | g |
| 3 | Ethyl cellulose 4 cps | 11.0 | g |
| 4 | Isopropyl alcohol | 175 | mL |

The modified release niacin formulation was prepared by the following process:

Niacin was sifted through a #20 sieve and microcrystalline cellulose (MCC) was sifted through a #40 sieve. Sifted materials were then loaded in to rapid mixer granulator (RMG) bowl and mixed for 5 minutes using impeller RPM at 75 RPM to provide a dry mix. Ethylcellulose powder was dissolved in isopropyl alcohol to provide the granulation solution. The granulation solution was added to the dry mix and the resulting mixture granulated in the RMG.

The wet granules were dried in a fluid bed drier (FBD) at 55°±2° C. Semi dried granules were passed through a #20 mesh and further dried in a fluid bed drier (FBD) until the loss on drying (LOD) at 105° C. was less than 2.0% (LOD found 1.26%). The dried granules were then passed through a #30 mesh.

The granules were then lubricated with microcrystalline cellulose and hydrogenated vegetable oil. The resulting lubricated granules were then compressed into tablets having an average weight of 630 mg. The compressed tablets were coated using an ethylcellulose solution in a conventional coating pan. A coating of about 2% was achieved.

Several batches were prepared. The dissolution profile for the batches is provided below. Dissolution was performed using 900 mL of aqueous media in a dissolution apparatus using a basket operated at about 37° C. being stirred at 100 rpm.

Dissolution Profile of the Tablets from Example 1:

| | Batch 1 | Batch 2 | Batch 3 | Batch 4 |
|---|---|---|---|---|
| Time | Cumulative | Cumulative | Cumulative | Cumulative |
| (Hrs.) | % release | % release | % release | % release |
| 1 | 26.96 | 9.13 | 8.41 | 18.22 |
| 2 | 44.33 | 16.71 | 12.42 | 21.92 |
| 3 | 50.98 | 23.66 | 17.19 | 26.79 |
| 4 | 59.27 | 28.56 | 22.23 | 32.40 |
| 6 | 68.77 | 37.58 | 29.98 | 41.66 |
| 9 | 80.78 | 48.48 | 39.30 | 52.75 |
| 12 | 82.47 | 55.58 | 50.04 | 60.79 |
| 16 | 89.27 | 65.17 | 60.57 | 68.75 |
| 20 | 98.81 | 76.03 | 69.06 | 76.92 |
| 24 | 100.35 | 82.38 | | 79.14 |

### Example 2

The general procedure of Example 1 was followed except for the following differences: niacin is 1000 mg/tablet. The excipients were adjusted accordingly. The dissolution profile for the tablets from Example 2 are provided below. Dissolution was performed using 900 mL of aqueous media in a dissolution apparatus using a basket operated at about 37° C. being stirred at 100 rpm.

Dissolution Profile of the Tablets from Example 2:

| | Cumulative % |
|---|---|
| Time (Hrs.) | Released |
| 1 | 9.43 |
| 2 | 22.05 |
| 3 | 31.05 |
| 4 | 37.40 |
| 6 | 47.51 |
| 9 | 59.46 |
| 12 | 67.99 |
| 16 | 77.76 |
| 20 | 83.07 |
| 24 | 88.99 |

### Example 3

The general procedure of Example 1 was followed with the relevant differences arising from the differences in the niacin-containing mixture as presented below.

| | Quantity |
|---|---|
| | for Trial |
| Ingredients | (g.) |
| Niacin | 500.00 |
| PVP K-30 | 15.00 |
| Ethyl cellulose 4 cps | 15.00 |
| Isopropyl alcohol | 785.00 |

The dissolution profile for the tablets from Example 3 are provided below. Dissolution was performed using 900 mL of aqueous media in a dissolution apparatus using a basket operated at about 37° C. being stirred at 100 rpm.

The Dissolution Profile of the Tablets from Example 3:

| | Cumulative | Cumulative |
|---|---|---|
| | % release | % release |
| Time (Hrs.) | Coated | Uncoated |
| 1 | 7.89 | 24.96 |
| 2 | 14.94 | 40.24 |
| 3 | 20.24 | 48.66 |
| 4 | 25.91 | 55.99 |
| 6 | 32.58 | 68.45 |
| 9 | 42.12 | 81.11 |
| 12 | 50.27 | 89.03 |
| 16 | 58.37 | 95.93 |
| 20 | 66.46 | 97.62 |
| 24 | | |

### Example 4

The general procedure of Example 1 was followed with the following differences: the niacin-containing granules were coated with ethylcellulose solution using bottom spray coating in fluid bed coating unit. The composition for coating the granules is given below:

| Ingredients | Qty. for trial | |
|---|---|---|
| Niacin granules | 100.00 | g |
| Ethylcellulose 4 cps | 3.0 | g |
| Isopropyl alcohol | 40 | mL |

The coated granules were compressed into tablets. The compressed tablets were then coated with HPMC in a conventional coating pan to achieve a coating of about 5%. These resulting coated tablets were then further coated using ethylcellulose to achieve a coating of about 4%.

The dissolution profile for the tablets from Example 4 are provided below. Dissolution was performed using 900 mL of aqueous media in a dissolution apparatus using a basket operated at about 37° C. being stirred at 100 rpm.

The Dissolution Profile of the Tablets from Example 4:

| Time (h) | Trial 1 | Trial 2 | Trial 3 |
|---|---|---|---|
| 1 | 18.16 | 3.53 | 2.92 |
| 2 | 27.89 | 7.43 | 6.12 |
| 3 | 38.56 | 13.78 | 11.62 |
| 4 | 44.25 | 21.23 | 18.15 |
| 6 | 52.36 | 36.52 | 30.82 |
| 9 | 63.23 | 51.74 | 48.03 |
| 12 | 71.71 | 64.58 | 58.88 |
| 16 | 78.52 | 66.29 | 62.91 |
| 20 | | | |
| 24 | | | |

### Example 5

Niacin 500 mg tablets and caplets (i.e., capsule shaped tablets) were prepared according to the process of Example 1 using the niacin-containing mixture given below.

| Sl. No. | Ingredients | Quantity for Trial (g.) |
|---|---|---|
| 1 | Niacin | 500.00 |
| 2 | MCC powder | 53.00 |
| 3 | Ethylcellulose 4 cps | 7.00 |
| 4 | PVP K-30 | 10.0 |
| 5 | Isopropyl alcohol | q.s. |
| 6 | Lubritab | 5.00 |

The caplets and tablets were coated to provide a coating in an amount of about 3.7%. The coating consisted of ethyl cellulose (4 cps) and Opadry clear at a ratio of 60:40 ratio. Diethyl phthalate in an amount of 25% w/w relative to ethyl cellulose was used as a plasticizer.

The dissolution profile for the tablets and caplets from Example 5 are provided below. Dissolution was performed using 900 mL of aqueous media in a dissolution apparatus using a basket operated at about 37° C. being stirred at 100 rpm.

The Dissolution Profile of the Tablets and Caplets from Example 5:

| | Coated | Coated |
|---|---|---|
| Time | Caplet | Tablet |
| 1 | 13.04 | 9.03 |
| 2 | 19.75 | 18.03 |
| 3 | 27.54 | 25.99 |
| 4 | 34.81 | 32.70 |
| 6 | 45.82 | 43.21 |
| 9 | 59.09 | 56.64 |
| 12 | 71.85 | 66.89 |
| 16 | 81.38 | 77.54 |
| 20 | 91.65 | 85.84 |
| 24 | 99.89 | 91.58 |

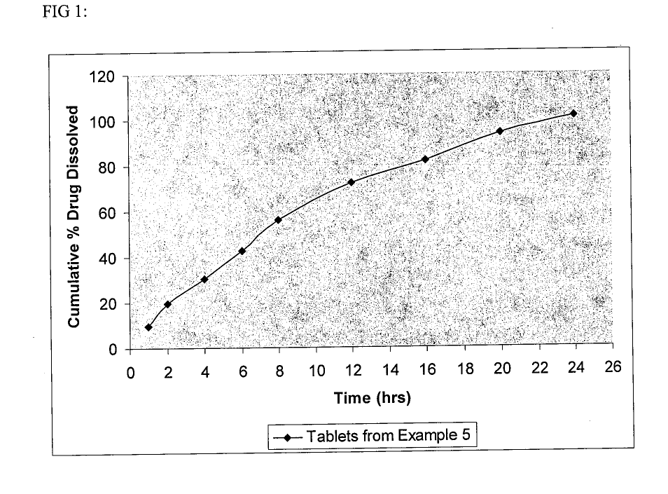

FIG. 1 provides a graphical representation of the in-vitro dissolution profile of the 500 mg coated tablet of Example 5.

### Example 6

A modified release niacin formulation was prepared from a niacin-containing mixture having the following composition:

| | | Quantity. for trial |
|---|---|---|
| Sl. No. | Ingredients | (kg.) |
| 1 | Niacin | 18.00 |
| 2 | Microcrystalline cellulose | 1.764 |
| | (Avicel pH 101) | |
| 3 | Ethylcellulose 4 cps | 0.831 |
| 4 | Povidone K-30 | 0.360 |
| 5 | Hydrogenated vegetable oil | 0.180 |
| | (Type 1) (Lubritab) | |
| 6 | Diethyl phthalate | 0.109 |
| 7 | Opadry clear 02B29055 | 0.290 |

The modified release niacin formulation was prepared by the following process:

Niacin was sifted through a #20 sieve and microcrystalline cellulose was sifted through a #40 sieve. Sifted materials were then loaded into the bowl of a rapid mixer granulator (RMG) and mixed for 10 minutes using impeller RPM at 75 RPM to provide a dry mix. Ethylcellulose 4 cps and Povidone K-30 were dissolved in isopropyl alcohol to provide the granulation solution. The granulation solution was added to the dry mix and the resulting mixture granulated in the RMG.

The wet granules were dried in a fluid bed drier (FBD) at 55°±2° C. until the loss on drying (LOD) at 105° C. was less than 2.0% (LOD found 1.26%). The dried granules were then passed through a #20 mesh.

The granules were lubricated with hydrogenated vegetable oil. The resulting lubricated granules were then compressed into tablets having an average weight 1150 mg.

### Coating Solution Preparation:

a) In a stainless steel vessel ethylcellulose 4 cps was added to isopropyl alcohol with stirring. Diethyl phthalate was then added with stirring. The resulting mixture was stirred to provide a clear solution.

b) Opadry clear 02B29055 was added to a mixture of isopropyl alcohol and purified water under stirring and the resulting mixture stirred until a clear solution is obtained.

c) The ethylcellulose 4 cps solution was then added to the Opadry clear solution slowly with stirring. The resulting solution was mixed for about 5 minutes.

The compressed tablets were coated using above coating solution in a conventional coating pan. A coating of about 3.5% was achieved.

Several batches were prepared. The dissolution profile for the batches is provided below. Dissolution was performed using 900 ml of aqueous medium in a dissolution apparatus using a basket operated at about 37° C., being stirred at a speed of 100 rpm.

### Dissolution Profile of the Tablets Prepared According to the Procedure of Example 6:

| Time (Hrs.) | Trial 86 |
|---|---|
| 1 | 9.62 |
| 2 | 16.48 |
| 3 | 24.30 |
| 4 | 30.22 |
| 6 | 40.49 |
| 9 | 52.37 |
| 12 | 63.55 |
| 16 | 74.72 |
| 20 | 83.77 |

### Example 7

Modified release niacin formulations analogous to those of Examples 1-6 were prepared wherein the amount of niacin per unit dosage form was changed to contain 250 mg, 500 mg, or 750 mg of niacin. The amount of excipients may remain the same or adjusted slightly to accommodate formulation-related parameters such as flowability, compressibility, etc.

### Example 8

A niacin and lovastatin formulation was prepared using the following components:

| | | |
|---|---|---|
| SI. No. | Ingredient | Quantity for Trial |

Core

| | | | |
|---|---|---|---|
| 1 | Niacin | 343.00 | g. |
| 2 | Microcrystalline cellulose | 36.00 | g. |
| 3 | Plasdone K29/32 | 6.96 | g. |
| 4 | Ethyl Cellulose | 4.80 | g. |
| 5 | Hydrogenated vegetable oil | 2.04 | g. |
| 6 | Isopropyl alcohol | 60.00 | ml |

Coating Solution

| | | | |
|---|---|---|---|
| 7 | Ethyl Cellulose | 7.30 | g. |
| 8 | Isopropyl Alcohol | 300.00 | ml |
| 9 | Water | 50.00 | ml |
| 10 | Opadry clear 02B29055 | 4.88 | g. |
| 11 | Diethyl phthalate | 1.80 | g. |

Lovastatin Coating Solution

| | | | |
|---|---|---|---|
| 13 | Lovastatin Sodium | 6.00 | g. |
| 14 | Methanol | 300.00 | ml |
| 15 | Plasdone K29/32 | 3.00 | g. |
| 16 | Triethyl Citrate | 1.00 | g. |
| 17 | Purified Talc | 2.00 | g. |

Protection Layer Coating Solution

| | | | |
|---|---|---|---|
| 18 | Hydroxypropyl Methylcellulose | 7.00 | g |
| 19 | Methanol | 350.00 | ml |
| 20 | Triethyl Citrate | 1.40 | g. |
| 21 | Purified Talc | 1.40 | g. |

The niacin and lovastatin formulation was prepared by the following process:

Niacin was sifted through a #20 sieve and microcrystalline cellulose was sifted through a #40 sieve. Sifted materials were then loaded into the bowl of a rapid mixer granulator (RMG) and mixed for 5 minutes using impeller RPM at 75 RPM to provide a dry mix. Plasdone K29/32 and ethylcellulose powder were dissolved in isopropyl alcohol to provide the granulation solution. The granulation solution was added to the dry mix and the resulting mixture granulated in the RMG.

The wet granules were then dried in a fluid bed drier (FBD) at 55°±2° C. Semi dried granules were passed through a #20 mesh and further dried in a fluid bed drier (FBD) until the loss on drying (LOD) at 105° C. was less than 2.0% (LOD found 1.26%). The dried granules were then passed through #30 mesh.

The granules were then lubricated with hydrogenated vegetable oil. The resulting lubricated granules were then compressed into capsule shaped tablets. The compressed tablets were coated using an ethylcellulose solution in a conventional coating pan. A coating of about 2% was achieved.

Coating solution preparation: Ethyl cellulose (7.30 gm) was dissolved in isopropyl alcohol (300 ml) with continuous stirring. Opadry clear (4.88 gm) was dissolved in water (50 ml). Aqueous Opadry clear solution was added to the ethyl cellulose solution with stirring. To this mixture was added diethyl phthalate (1.80 gm) with stirring.

The coating solution was then spray coated onto the above capsule shape tablets using a conventional coating pan using a spray gun.

Lovastatin sodium coating solution: Lovastatin sodium (6.00 g.) was dissolved in methanol (300 ml). Plasdone K29/32 (3.00 g.) was added to the above lovastatin sodium solution under stirring. Triethyl citrate (1.00 g.) was then added. To the resulting solution was then added purified talc (2.00 g.) with stirring.

The lovastatin sodium coating solution was coated onto the above coated tablets by spray coating in a conventional coating pan using spray gun.

Protection layer coating solution: Hydroxypropyl methylcellulose (7.00 gm) was dissolved in methanol (350 ml). Triethyl citrate (1.40 gm) was added to the above solution with continuous stirring. Purified talc (1.40 gm) was then added to the above solution.

The protective coating layer was coated onto the above lovastatin coated tablets by spray coating in a conventional coating pan using spray gun.

Several batches were prepared. The dissolution profile for the batches is provided below. Dissolution was performed using 900 ml of 6.8 pH phosphate buffer in a dissolution apparatus using a basket operated at about 37° C., being stirred at a speed of 100 rpm.

### Dissolution Profile of the Tablets Prepared According to the Procedure of Example 8:

| | Niacin 1000 mg/ |
|---|---|
| Time | 20 mg lovastatin |
| Interval (Hrs) | Coated |
| 1 | 9.48 |
| 2 | 19.77 |
| 4 | 30.33 |
| 6 | 42.55 |
| 8 | 56.31 |
| 12 | 72.5 |
| 16 | 81.99 |
| 20 | 93.67 |
| 24 | 101.07 |

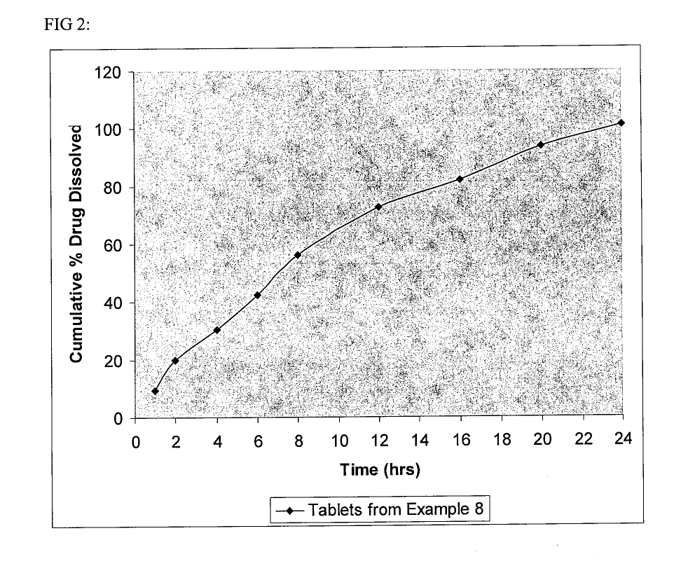

FIG. 2 provides a graphical representation of the in-vitro dissolution profile of the Niacin 1000 mg/lovastatin 20 mg coated tablet of Example 8.

### Example 9

Niacin and lovastatin formulations analogous to that of Examples 8 were prepared wherein the amount of niacin and lovastatin per unit dosage form was changed to contain 500 mg/20 mg, or 750 mg/20 mg, or 1000 mg/40 mg of niacin/lovastatin. The amount of excipients may remain the same or adjusted slightly to accommodate formulation-related parameters such as flowability, compressibility, etc.

The examples given above are merely illustrative and are not meant to be an exhaustive list of all possible embodiments, applications, or modifications of the invention. Various modifications of the described methods and compositions will be apparent to those skilled in the art without departing from the scope and spirit of the invention.

The disclosures of all references and publications cited above are expressly incorporated by reference in their entireties to the same extent as if each were incorporated by reference individually.

What is claimed is:

1. A modified release niacin formulation comprising:
a plurality of granules comprising a therapeutically effective amount of niacin, a pharmaceutically acceptable binder, and an excipient,
wherein the granules are compressed together to form a core, and
the core is coated with a release-modifying coating.

2. The formulation of claim 1, wherein the binder is present in an amount ranging from about 0.01% to about 30% by weight of the granules.

3. The formulation of claim 1, wherein the release-modifying coating is present in an amount ranging from about 0.5% to about 30% by weight of the core.

4. The formulation of claim 1, wherein the excipient is selected from the group consisting of sugars, carbohydrates, polyols, celluloses, microcrystalline cellulose, mono-calcium phosphate, di-calcium phosphate, tri-basic calcium phosphate, starch, sodium starch glycolate, crospovidone, croscarmellose sodium, magnesium stearate, lactose, stearic acid, maleic acid, a wax, colloidal silicon dioxide, talc, gelatin, polyethylene glycol, titanium dioxide, glyceryl behenate, fats, emulsifiers, and mixtures thereof.

5. The formulation of claim 1, wherein the binder is selected from the group consisting of vinyl polymers, cellulosic polymers, acrylic polymers and copolymers, natural gums, synthetic gums, proteins, and carbohydrates.

6. The formulation of claim 5, wherein the vinyl polymer is selected from the group consisting of polyvinylpyrrolidone and polyvinyl alcohol; the cellulosic polymer is selected from the group consisting of HPMC, HEC, and HPC; the acrylic polymer and copolymer is selected from the group consisting of methacrylic acid polymers and ethyl acrylate-methylmethacrylate copolymers, the natural gum and synthetic gum is selected from the group consisting of guar gum, arabic gum, xanthan gum; and the carbohydrate is selected from the group consisting of gelatin and pectin.

7. The formulation of claim 1, wherein the niacin is present in an amount ranging from about 25.0 mg to about 2000 mg.

8. The formulation of claim 1, wherein the release-modifying coating is selected from the group consisting of cellulose-based polymers, acrylate polymers, and waxes.

9. The formulation of claim 8, wherein the cellulose-based polymer is selected from the group consisting of ethylcellulose, propylcellulose, hydroxymethylpropylcellulose, hydroxypropyl cellulose and the acrylate polymer is selected from the group consisting of polymers of methylmethacrylate and polymers of methacrylate.

10. The formulation of claim 9, wherein the release-modifying coating is ethylcellulose.

11. A process for making a modified release niacin formulation comprising:
a) combining niacin, a binder, and a pharmaceutically acceptable excipient to provide a niacin-containing mixture,
b) granulating the niacin-containing mixture to provide niacin-containing granulates;
c) compressing said niacin-containing granulates to provide a niacin-containing core; and
d) coating the niacin-containing core with a release-modifying coating to provide a release-modified coated core.

12. The method of claim 11, further comprising coating the niacin-containing granulates with a release-modifying coating before they are compressed.

13. The method of claim 11, wherein the release-modifying coating is present in an amount ranging from about 0.5% to about 10% by weight of the core.

14. The method of claim 11, wherein granulating is wet granulating using a non-aqueous solvent.

15. The method of claim 14, wherein the non-aqueous solvent is selected from the group consisting of ethanol, isopropanol, acetone, methanol, and mixtures thereof.

16. The method of claim 11, wherein the release-modifying coating is selected from the group consisting of cellulose-based polymers, acrylate polymers, and waxes.

17. The method of claim 11, wherein the cellulose-based polymer is selected from the group consisting of ethylcellulose, propylcellulose, hydroxymethylpropylcellulose, hydroxypropyl cellulose and the acrylate polymer is selected from the group consisting of polymers of methylmethacrylate and polymers of methacrylate.

18. The pharmaceutical composition of claim 1, wherein the core that is coated with the release-modifying coating is further coated with a second coating, wherein the second coating comprises a pharmaceutically active agent.

19. The pharmaceutical composition of claim 18, wherein the pharmaceutically active agent is selected from the group consisting of cardiovascular agents and non-steroidal anti-inflammatory agents.

20. The pharmaceutical composition of claim 19, wherein the pharmaceutically active agent is lovastatin.

21. The process of claim 11, further comprising:
e) coating the release-modified coated core with a second coating, wherein the second coating comprises a pharmaceutically active agent.
